(19) United States
(12) Patent Application Publication
(10) Pub. No.: US 2016/0066994 A1
(43) Pub. Date: Mar. 10, 2016

(54) Non-Invasive Method for Slimming a Human Body Using Two Wavelngths of Laser Energy Concurrently

(71) Applicant: Erchonia Corporation, McKinney, TX (US)

(72) Inventor: Steven C. SHANKS, McKinney, TX (US)

(73) Assignee: Erchonia Corporation, McKinney, TX (US)

(21) Appl. No.: 14/481,825

(22) Filed: Sep. 9, 2014

(51) Int. Cl. A61B 18/20

(52) U.S. Cl. CPC A61B 18/203; A61B 2018/2065

(57) ABSTRACT

A noninvasive method of slimming a patient's body by concurrently applying laser energy of two different wavelengths to the patient. In the preferred embodiment, the laser treatment uses one wavelength of about 635 nm and another wavelength of about 532 nm. Preferably the laser energy is applied using a circular beam spot created by a line swept through a 360° circle. By using two wavelengths concurrently in the treatment regimen, the patient avoids the inconvenience and expense of the trial-and-error approach of undergoing a complete treatment regimen unsuccessfully with a first wavelength, and then subsequently undergoing a second complete treatment regimen using a second wavelength.

### FIELD OF INVENTION

This invention relates to a method for non-invasive, non-destructive shaping and contouring of a human body by external means. In particular, this invention relates to the concurrent application of laser energy of two wavelengths to targeted external regions of a patient's body to slim the patient's body.

### BACKGROUND

There is a great demand to be slimmer. The tried and true method of taking in fewer calories than a person expends results in weight loss, and the resultant slimming effect occurs over the body as a whole. For more targeted contouring, many people resort to the cosmetic surgical procedure known as liposuction, wherein excess adipose tissue, also known as fat, is suctioned from the body of a patient. The typical purpose of the liposuction procedure is to leave the patient thinner in desired areas, with aesthetically more appealing body contours. For example, liposuction is often performed on patients to remove excess fat in the abdominal, buttock, thigh, breast or arm regions of the body.

Liposuction is performed by inserting a narrow tube, or cannula, through a tiny incision in the skin into the subcutaneous fatty tissue. The cannula is repeatedly pushed then pulled through the fat layer, separating and puncturing the fat cells and suctioning them out. Suction action through the cannula is provided by a vacuum pump or a large syringe. The procedure carries with it some risks and side effects. Due to the physical damage induced, the procedure can damage nerves, lymphatics and vasculature in the surrounding area, often resulting in significant loss of blood as the blood is vacuumed out with the fat and the formation of seroma due to damaged lymphatic channels. In addition, the post-procedure recovery period is long and often accompanied by a great deal of inflammation, bruising and concomitant pain.

Since the liposuction technique was first developed there have been many improvements to techniques for contouring the body, with the goal of making the surgery less dangerous for the patient, reducing the negative aspects of the post-operative recovery period, and making it more commercially viable for the practitioner who treats the patient.

Non-invasive methods of body contouring are preferred over invasive methods to minimize trauma to the patient, reduce the risk of infection, and speed up recovery time, among other reasons. To avoid invasive procedures, electromagnetic energy, such as microwave, ultrasound or radio frequency radiation, has been used to reduce fat. In U.S. Pat. No. 5,507,790 issued to Weiss, a method is described in which a medicament is applied to a patient's skin where fat removal is desired and focused electromagnetic energy is applied to the same work site to heat the fatty tissue and increase fat lipolysis. In U.S. Pat. No. 5,143,063, Fellner takes this method even farther, applying sufficient electromagnetic radiation to destroy the fat cells. Yet another method is to inject an intumescing solution below the skin and apply electromagnetic energy externally to the body. These procedures are disadvantageous in that they utilize such high energy sources that they excessively heat the surrounding tissue, which can result in damage to the tissue and pain. Again, recovery time is significant.

In a more recent innovation, a procedure was developed by a group including one of the inventors of this method which uses red LLLT alone, to contour the body by reducing fat. The procedure is described in U.S. Pat Pub. 2005/0203594 and involves using a device having lasers of less than 1 W to apply one or more treatments of 635 nm laser energy externally to the patient to release at least a portion of the intracellular fat into the interstitial space through a transitory pore in the fat cell. Upon sufficient doses of this low-level laser energy at 635 nm, the cell membrane is believed to be momentarily disrupted, releasing the intracellular fat into the interstitial space. Upon cessation of the energy application, the pore closes and the cell membrane returns to contiguity. The treated and surrounding tissue is not heated and not damaged, and the patient feels no sensation during the application of the 635 nm laser energy. The released fat is removed from the patient's body through one or more of the patient's normal bodily systems. Many patients respond very well to the treatments and the method revolutionized the market for body contouring because it reduces fat with no trauma to the patient. Curiously, however, some patients did not respond as well—or at all—to the treatment with red laser energy.

Historically the LLLT industry has believed that using lasers with wavelengths shorter than about 632 nm would fail to non-traumatically shape a patient's body. One reason is that it is believed that the shorter wavelengths would not penetrate the skin deep enough to reach the fat cells and other tissues needed to attain shaping. For example, U.S. Pat. No. 7,771,374 issued to Slatkine, discloses that melanin and blood in the skin do not allow light at 405 nm, 514 nm or 585 nm to penetrate deep into the skin due to strong absorption. Instead, the patent discloses using a vacuum to expel blood from the treatment area to improve treatment of the tissue.

Lasers emit electromagnetic energy, which can be described by frequency, wavelength, or energy. Laser diodes with shorter wavelengths have a higher energy than laser diodes with longer wavelengths. Another reason that it was believed that the shorter wavelengths would fail to non-traumatically reduce fat was because in order to penetrate the skin to a deep-enough depth, the higher-energy wavelengths would heat the surrounding tissue through which the laser penetrated, traumatizing the patient. Laser devices emitting wavelengths shorter than about 632 nm lasers are known in the art, but only for ablative treatments. To cool the radiated skin and minimize trauma to the patient, complicated cooling components were invented to reduce the heat generated by the shorter wavelength therapeutic lasers. For example, in U.S. Pat. Pub 2008/0294153, Altshuler describes a cooling system for a green laser light used as a thermal treatment to remove a red portion of a tattoo by causing the death of the cells containing the tattoo ink particles by rupture or apoptosis.

Yet another reason that it was believed that the shorter wavelengths would fail to non-traumatically shape a targeted area of the body is that any wavelength other than about 635 nm would be ineffective for stimulating the cell to open the transitory pore to release fat. Consequently, only devices emitting red laser energy had been used for shaping the body until the inventors hereof pioneered the use of laser energy at less than 632 nm for body contouring.

Interestingly, while some patients do not respond to laser therapy for fat reduction using a wavelength in the red range, they respond to laser therapy having a wavelength in the green range. Similarly, others do not respond to laser therapy using a wavelength in the green range, but find success in fat reduction using with laser therapy using a wavelength in the red range. If a patient is treated with a wavelength to which he is non-responsive, it not only fails to shape the patient's body, but the lack of body contouring is disappointing to the patient and the therapist. If would be desirable to treat a patient with a method that increases the likelihood the patient's fat will be reduced.

Therefore, an object of this invention is to provide a non-invasive method of low-level laser therapy that increases the likelihood of reducing fat of the patient. It is another object of the low-level laser therapy method to increases the likelihood of slimming desired areas of the patient. Another object is to provide such a method that does not destroy fat cells or otherwise damage surrounding tissue or structures. It is another object to provide a non-invasive method of slimming a human body using laser energy of two or more wavelengths shorter than 632 nm.

### SUMMARY OF THE INVENTION

This invention is a noninvasive method of slimming a patient's body by concurrently applying laser energy of two different wavelengths to the patient. In the preferred embodiment, the laser treatment uses one wavelength of about 635 nm and another wavelength of about 532 nm. Preferably the laser energy is applied using a circular beam spot created by a line swept through a 360° circle. By using two wavelengths concurrently in the treatment regimen, the patient avoids the inconvenience and expense of an unsuccessful treatment regimen at one wavelength followed, followed by a second complete treatment regimen using a second wavelength.

### BRIEF DESCRIPTION OF THE DRAWINGS

FIG. 1 illustrates a patient 10 being treated using the preferred embodiment of the present invention with eight sweep beam spots of laser energy.

FIGS. 2A-K illustrate the start and stop times of several instances of the first wavelength (top line) and the second wavelength (bottom line).

FIG. 3 illustrates part of a prior art laser device for creating a circular beam spot by sweeping a line swept through a 360° circle.

### DETAILED DESCRIPTION OF THE INVENTION

Lasers emit electromagnetic energy, which can be described by frequency, wavelength, or energy. Laser diodes with shorter wavelengths have a higher energy than laser diodes with longer wavelengths. See Table 1:

| Wavelength | Energy | % difference |
|---|---|---|
| (nm) | (eV) | over 635 nm |
| 635 (red) | 1.82 | — |
| 532 (green) | 2.24 | 23% |
| 440 (violet) | 2.84 | 56% |
| 405 (violet) | 3.09 | 68% |

Despite heretofore long-recognized reasons that using laser energy having wavelengths shorter than 635 nm to treat patients for fat reduction would be ineffective—or worse, dangerous—the present invention is a method for slimming a patient's body non-traumatically by using laser energy having one or more of these shorter wavelengths, preferably green or violet laser light, in combination with red laser light. In the preferred embodiment, one of the laser treatments uses a wavelength of about 635 nm and another of the laser treatments uses a wavelength of about 532 nm.

In describing the invention herein one of the wavelengths is often described as the “first” wavelength and the other wavelength as the “second” wavelength. As used herein, “first” and “second” do not necessarily refer to the chronological order in which the laser treatments are applied, but instead simply as adjectives to distinguish each wavelength from another. For example, if the method employs a laser treatment with a first wavelength in the red range and a second wavelength in the green range, then the chronological order of the colors as applied to the patient can be red before green; green before red; or red and green at the same time.

To increase the likelihood of reducing fat of every patient, the present invention uses two wavelengths of laser energy with the expectation that the patient will respond to at least one of the wavelengths. By using two wavelengths concurrently in the treatment regimen, the patient avoids the inconvenience and expense of the trial-and-error approach of undergoing a complete treatment regimen unsuccessfully with a first wavelength, and then subsequently undergoing a second complete treatment regimen using a second wavelength.

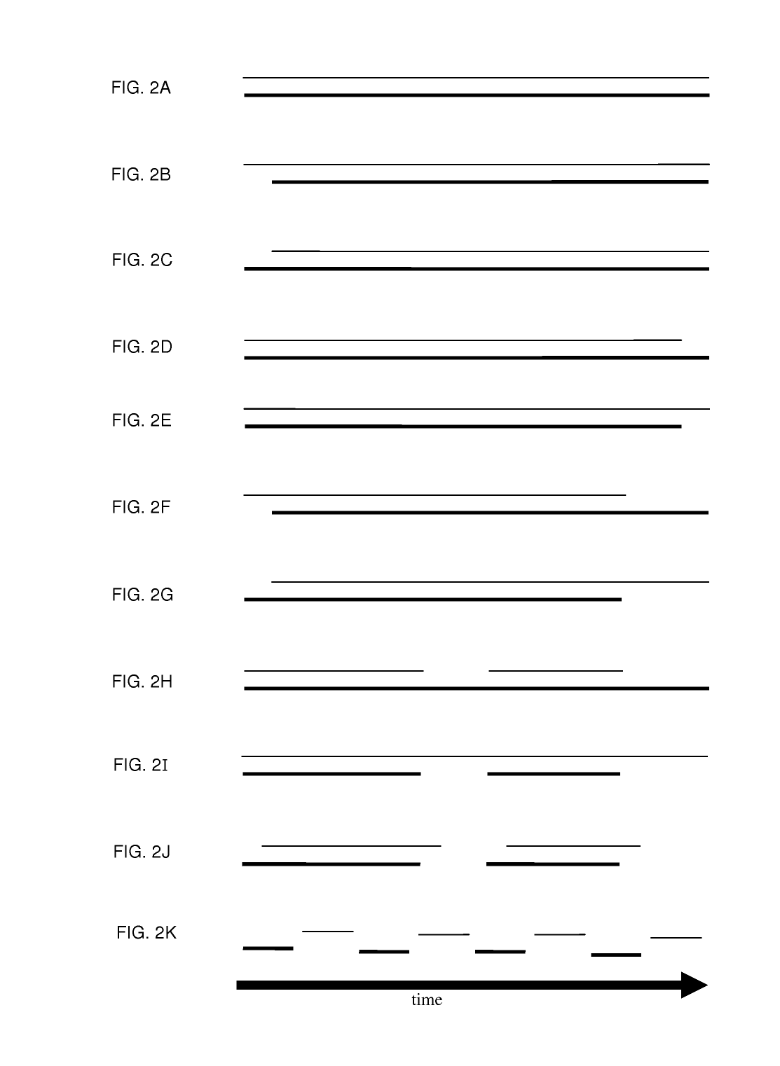

The wavelengths of laser energy are supplied concurrently, which as used herein means that during the period of time the first wavelength is applied to the patient, the second wavelength is applied for some period of time too. That is, the treatment periods of the first and second wavelengths overlap for all or some portion of the treatment time. FIGS. 2A-K illustrate the start and stop times of the first wavelength (top line) and the second wavelength (bottom line). The first and second wavelengths of laser energy may start at the same time (e.g., FIG. 2A) or different times (e.g., FIGS. 2B, 2C, 2F, 2G, 2J and 2K); may end at the same time (e.g., FIGS. 2A, 2B, and 2C) or different times (e.g., FIGS. 2D, 2E, 2F, 2G, 2H, 2I, 2J and 2K). The treatment period of each wavelength may be broken into a series of smaller portions, with or without laser energy being applied between the portions. See, for example, FIGS. 2I, 2J and 2K. The length of the lines in FIGS. 2A-K is not necessarily to scale, does not indicate the absolute period of treatment time, and does not necessarily indicate the amount of time the first wavelength is being applied relative to the second wavelength. In addition to starting and stopping at different times, the laser energy may be pulsed, and the pulse frequency of each wavelength may be the same or different from the pulse frequency each other wavelength.

The laser energy is provided by two or more low-level laser devices, known in the art as cold lasers, such as the inventions described in U.S. Pat. No. 6,013,096 issued to Tucek and U.S. Pat. No. 6,746,473, issued to Tucek and Shanks. The laser energy sources in these devices use semiconductor diode lasers which are available commercially in a broad range of wavelengths between 405-1500 nm. Each laser device may have one or more laser energy sources.

Different therapy regimens require laser energy sources of different power output. The preferred laser diodes have a maximum output of 1 W of power; more preferably at least 5 mW output and most preferably at least 17 mW output. For fat reduction, more than 5 mW output per laser energy source is needed in order to have enough energy to create the transitory pore. Below 5 mW, the fluence is insufficient to open the pore through which the fat is released, so there is no contouring. There are no known contraindications of low level laser therapy using laser energy sources of up to 1 W, although it is prudent for the patent and therapist to wear appropriate safety glasses. Laser energy sources of various other wattages may also be employed to achieve the desired laser energy for the given regimen. The laser energy is applied externally to the patient at a targeted area in a dose rate that causes no detectable temperature rise of the treated tissue.

The dosage of laser energy required to achieve slimming will vary depending on the thickness of the patient's skin, thickness of fatty tissue, and other biological factors peculiar to each patient. While a person skilled in the art will be able to determine the amount of energy needed to slim the patient by comparing initial and subsequent measurements, applied energy can also be determined by the applied fluence. A 20 minute treatment using a 500 mW laser energy source has a total fluence of 600 joules per laser and fluence by area of 2.587 joules per square centimeter per treatment. A 20 minutes treatment using a 17 mW laser energy source has a total fluence is 21 joules per laser and a fluence by area of 0.091 joules per centimeter squared per treatment. Preferably the fluence is a range of about 1.05 to 30 Joules per square centimeter per minute.

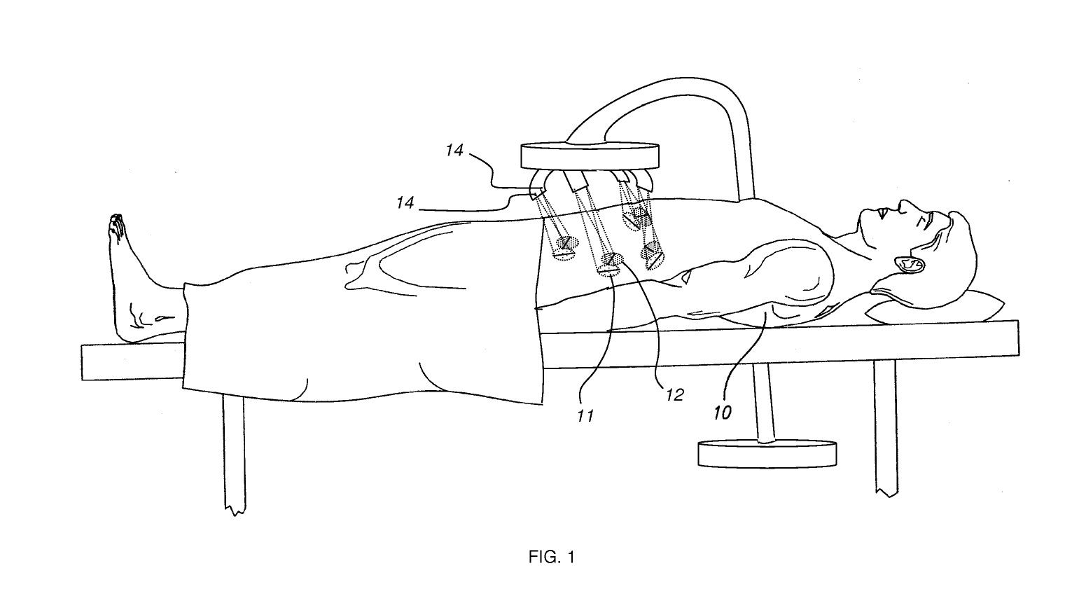

In the preferred embodiment, each wavelength of laser energy is applied using a separate circular beam spot created by a line swept through a 360° circle. FIG. 1 illustrates a patient 10 being treated using the preferred embodiment of the present invention with four sweep beam spots of a first wavelength 11 and four sweep beam spots of a second wavelength 12, for a total of eight beam spots. The laser energy is targeted at the patient's belly area. In FIG. 1 the beam spots of the first wavelength and the second wavelength do not overlap when they are applied the target area. In alternative embodiments the beam spots of the first wavelength and the second wavelength may overlap when they are applied the target area. The overlap may be partial or total.

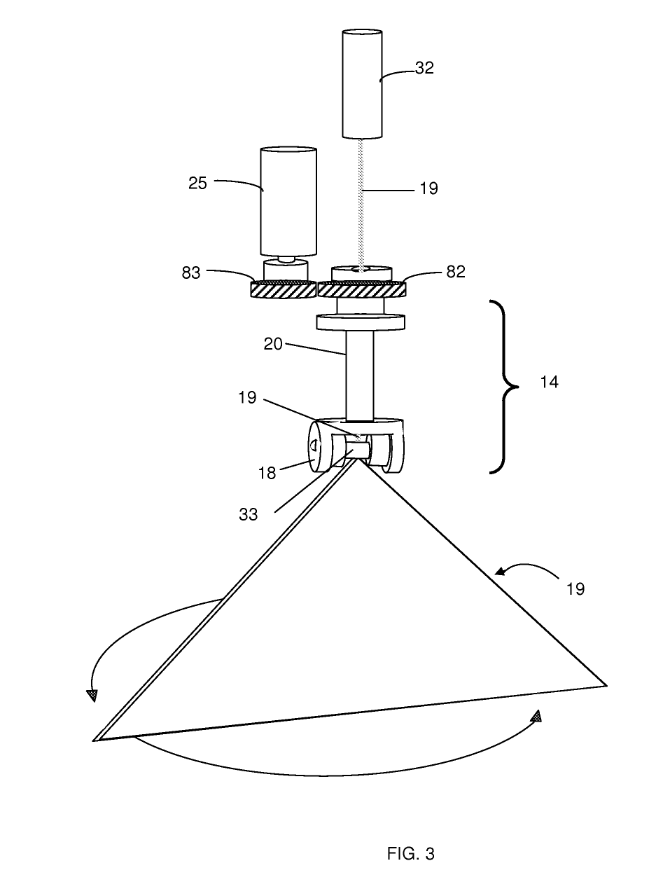

Devices for creating each sweep beam spot are disclosed in U.S. Pat. No. 7,947,067, entitled “Scanning Treatment Laser with Sweep Beam Spot and Universal Carriage,” which is incorporated herein by reference. FIG. 3 shows a scanning laser device disclosed in that patent that sweeps a circular beam spot. The device employs a universal carriage 18 that holds a refractive optical element 33. A line 34 is generated when a laser beam 19 from a semiconductor diode 32 strikes the optical element 33. The carriage rotates and the line rotates, too, becoming, in essence, a rotating diameter of a circular beam spot. If the carriage is rotated through 360°, the line sweeps through a complete circle. With electronic or computerized control, the carriage automatically rotates continuously and very quickly, causing the laser beam to appear to create a substantially circular beam spot on the patient's skin. See FIG. 1. The shape, however, is actually the result of the scanning light diameter sweeping from location to location at a speed that makes the motion nearly imperceptible to the human eye. The longer the line, the larger the beam spot.

To apply two wavelengths of laser energy concurrently in circular beam spots to the patient, a separate scanning laser device 14 is used for each wavelength. To apply four sweep beam spots of a first laser energy 11 and four sweep beam spots of a second laser energy 12, as shown in FIG. 1, eight scanning laser devices 14 are used

Often patients are satisfied with the slimming results in one therapy session, however each patient may also be subjected to multiple sessions over several weeks' time. In one example, a patient was treated with laser energy once a week for six weeks using a first laser wavelength of 635 nm emitted from a first 17 mW semiconductor laser diode and a second laser wavelength of 532 nm from a second 17 mW semiconductor laser in side-by-side swept beam spots. Each treatment session was 20 minutes long, and the red laser energy and green laser energy were applied concurrently with the same start and stop times. The patient lost twice as much fat, as indicated by circumference measurements made of the targeted areas, as what the patient had seen before with similar treatment using only 635 nm laser energy.

While there has been illustrated and described what is at present considered to be a preferred embodiment of the present invention, it will be understood by those skilled in the art that various changes and modifications may be made, and equivalents may be substituted for elements thereof without departing from the true scope of the invention. Therefore, it is intended that this invention not be limited to the particular embodiment disclosed as the best mode contemplated for carrying out the invention, but that the invention will include all embodiments falling within the scope of the appended claims.

1. A method of slimming a patient at a targeted area, the method comprising:
a. applying first laser energy having first wavelength externally to the patient at the targeted area; and
b. applying second laser energy having second wavelength externally to the patient at the targeted area;
c. wherein the first and second laser energy are applied concurrently; and the first and second wavelengths are different.

2. The method according to claim 1 wherein the first wavelength is green and the second wavelength is red.

3. The method according to claim 1 wherein the first wavelength is about 532 nm and the second wavelength is about 635 nm.

4. The method according to claim 1 wherein the first wavelength is about 440 nm and the second wavelength is about 635 nm.

5. The method according to claim 1 wherein the first wavelength is about 405 nm and the second wavelength is about 635 nm.

6. The method according to claim 1 wherein the first laser energy and second laser energy are applied in circular beam spots.

7. The method according to claim 6 wherein the first and second circular beam spots do not overlap.

8. The method according to claim 6 wherein there are two or more beam spots of the first laser energy and two or more beam spots of the second laser energy.

9. The method according to claim 1 wherein the first and second laser energy are applied simultaneously.

10. The method according to claim 1 wherein the first and second laser energy are applied for about the same length of time.

11. The method according to claim 1 wherein in the first laser energy and the second laser energy are applied in a dose rate that causes no detectable temperature rise of the treated tissue.

12. The method according to claim 1 wherein in the first laser energy is supplied by a first laser energy source having a maximum output of 5-500 mW and the second laser energy is supplied by a second laser energy source having a maximum output of 5-500 mW.

13. A method of slimming a patient, the method comprising:
a. applying a first laser energy having a first wavelength externally to the patient at a targeted area by sweeping the first laser energy through a 360° circle to create a first circular beam spot; and
b. concurrently applying a second laser energy having a second wavelength externally to the patient at the targeted area by sweeping the second laser energy through a 360° circle to create a second circular beam spot;
c. wherein the first and second wavelengths are different.

14. The method according to claim 11 wherein the first and second circular beam spots do not overlap.

15. The method according to claim 11 wherein there are two or more beam spots of the first laser energy and two or more beam spots of the second laser energy.

16. The method according to claim 11 wherein sweeping the first laser energy comprises:
a. disposing a first optical element in a first rotatable carriage;
b. striking the first optical element with the first laser energy;
c. rotating the first carriage through 360°; and
wherein sweeping the second laser energy comprises:
d. disposing a second optical element in a second rotatable carriage;
e. striking the second optical element with the fi second rst laser energy;
f. rotating the second carriage through 360°.

17. The method according to claim 11 wherein the first wavelength is about 532 nm and the second wavelength is about 635 nm.

18. The method according to claim 11 wherein the first wavelength is about 440 nm and the second wavelength is about 635 nm.

19. The method according to claim 11 wherein the first wavelength is about 405 nm and the second wavelength is about 635 nm.

20. The method according to claim 11 wherein in the first laser energy is supplied by a first laser energy source having a maximum output of 5-500 mW and the second laser energy is supplied by a second laser energy source having a maximum output of 5-500 mW.
